(19) United States
(12) Patent Application Publication
(10) Pub. No.: US 2003/0157586 A1
(43) Pub. Date: Aug. 21, 2003

(54) Device and method for conducting cellular assays using multiple fluid flow

(76) Inventors: Martin Bonde, San Jose, CA (US); Michael Beyer, San Jose, CA (US); Thomas Ahl, Cupertino, CA (US)

Correspondence Address: REED & EBERLE LLP, 800 MENLO AVENUE, SUITE 210, MENLO PARK, CA, 94025, US

(21) Appl. No.: 10/080,901

(22) Filed: Feb. 21, 2002

(51) Int. Cl. C12Q 1/02; C12M 1/22; C12N 5/00

(52) U.S. Cl. 435/29; 435/402; 435/305.2

(57) ABSTRACT

The invention relates to a device for exposing a substrate surface to at least one fluid. The device comprises a substrate having a surface containing a contiguous target region and a cover plate. A plurality of fluid-transporting features is present in a cover plate surface, and the features are separated by at least one partitioning wall representing an integral portion of the cover plate. The features fluidly communicate with at least one outlet, and each feature fluidly communicates with an inlet. The cover plate surface is positioned in fluid-tight contact with the substrate surface such that the at least one partitioning wall contacts the contiguous target region. As a result, each feature, in combination with the substrate surface, forms a flow passage containing a distinct exposure zone on the target region. Also provided are methods for exposing a substrate surface to a plurality of cells and methods for detecting cell-cell interactions.

### TECHNICAL FIELD

The present invention relates to devices and methods that employ multiple fluid flows to effect rapid and efficient cell-based analysis. More specifically, the invention relates to the delivery of a plurality of cells to distinct exposure zones on a contiguous target region of a substrate surface through use of a plurality of fluid flows.

### BACKGROUND

There is a need for high-throughput cellular screening technology to provide critical information for the understanding of complex cell functions. Assays employing living cells, in particular, can provide data that may approximate in vivo animal and human experimentation. Cellular assays, for example, may be used to evaluate intercellular interactions as well as interactions between pharmacological compounds, and represent information-rich testing procedures.

Several approaches have been suggested for studying cell-cell interactions. Early in vitro models were based on the so-called Stamper-Woodruff assay. In this assay, a suspension of lymphocytes is placed on top of a thin section of rat or mouse tissue. The force of gravity brings the lymphocytes into contact with the tissue section. Once contact has been established, bound cells are fixed, visualized, and identified under a light microscope. See Stamper et al. (1976) J Exp Med 144(3):828-833. U.S. Pat. No. 6,010,845 to Poston describes a variation on the Stamper-Woodruff assay. A disadvantage of the Stamper-Woodruff assay format is that it does not mimic physiological conditions, relying instead upon gravity or centrifugal forces (as opposed to other significant influences commonly exhibited in vivo, such as fluid flow). In most cases, this assay format also does not allow for the easy visualization of cells using standard light microscopy techniques.

A number of cytometers and other apparatuses for conducting cellular assays are commercially available. For example, apparatuses for cellular assays that employ a flow of fluid to transport reagents or cells over immobilized cells are available from GlycoTech Corporation in Rockville, Md. A significant drawback to such approaches, however, is that they generally involve the use of a relatively large quantity of cells.

A number of patents describe the use of cellular arrays. For example, U.S. Pat. Nos. 5,976,826 and 5,776,748 to Singhvi are related patents, each directed to a device for adhering at least one cell in a specific and predetermined pattern. The device includes a plate that defines a surface as well as a plurality of cytophilic islands, the surfaces on which cells may adhere. The cytophilic islands, formed from a self-adhesive monolayer, are isolated by contiguous cytophobic regions to which cells do not adhere. The cytophobic regions may be sufficiently wide to prevent cells adhered to the cytophilic islands from contacting each other, except via formation of cellular bridges that lie above (and thus free of adhesive contact with) the cytophobic regions. The cytophobic regions may, alternatively, be sufficiently wide such that less than 10 percent of cells adhered to the cytophilic islands form bridges across said cytophobic regions and contact each other. U.S. Pat. No. 6,180,239 to Whitesides et al. describes that such an array may be formed by employing a stamp for imparting a pattern of the self-assembled monolayer of the molecular species on a surface.

U.S. Pat. No. 6,103,409 to Taylor describes a method for producing a cassette for cell screening. A base with a surface is provided, and a micropatterned chemical array is prepared. The micropatterned chemical array is modified to produce a modified micropatterned chemical array comprising multiple different cell binding locations on the surface of the base. The different cell-binding locations interact with different cell types, and each cell-binding location comprises a well. Once cells are bound to the modified micropatterned chemical array to produce an ordered array of cell types seeded on the wells, a fluid delivery system is provided for delivering a combinatorial library of reagents to the ordered array of cell types. The fluid delivery system is typical of many microfluidic devices in that it comprises a chamber that mates with the base containing the ordered array of cell types. The chamber comprises: (i) etched domains matching the wells on the surface of the base, and (ii) microfluidic channels that supply fluid to the etched domains.

Thus, if array technology is employed to carry out cellular assays, there must be a means to controllably deliver fluids or cells to different array feature locations. This may be carried out through known cytometry equipment, such as those that employ hydrodynamically focused flow. International Publication WO 00/56444, for example, describes a method for producing an interaction between a hydrodynamically focused liquid (or a component of the hydrodynamically focused liquid) and a selected region of a target surface. Cells may be immobilized on the target surface. The method involves providing a target surface that defines, in part, a liquid flow path that uses two guidance streams to direct a flow of a hydrodynamically focused liquid stream, which is then interposed between the liquid guidance streams over the selected region of the target surface. By adjusting the flow ratio of the guidance streams, the position of the focused liquid stream may be controlled. Thus, cells immobilized on the target surface may be selectively exposed to the focused liquid stream. While this method provides great accuracy with respect to positioning the hydrodynamically focused liquid, the method requires independent control over the flow rate of each stream. As the number of streams is increased, a relatively sophisticated and expensive flow control system is needed to ensure accuracy and repeatable stream positioning. Similarly, the methods and devices described in: U.S. Serial No. 60/286,819 (“A Method for Interacting a Product Substance with a Substance Retained on a Surface”), inventors Beyer, Krühne and Ahl; U.S. Serial No. 60/285,494 (“Sample Introduction into Apparatus for Hydrodynamically Focused Flow”), inventors Beyer and Krühne; U.S. Serial No. 60/286,550 (“Methods for Directing a Hydrodynamically Focused Flow of Liquid over a Topologically Variable Surface”), inventors Beyer, Krühe and Bonde; and U.S. Pat. No 6,200,814 to Malmqvist et al. suffer from the same drawback.

Thus, known cellular assay technology suffers from the drawback that sophisticated cell placement equipment, complex fluid handling equipment, or both are required. As a result, known miniaturized cellular assay technology either exhibits a low throughput, high cost, or both. Although cellular array technology reduces the quantity of cells and/or reagent required to carry out cellular assays, known assays involving cellular array technology typically require precise alignment between the cellular array and the fluid handling equipment. This, in turn, increases the complexity and cost of cellular assays.

Accordingly, there is a need for alternative methods and devices that are capable of efficiently conducting cellular assays. In particular, such methods and devices are needed to assess cell-cell interactions. Such methods and devices should allow for high-throughput screening to be conducted with ease and without requiring the expense and/or complexity associated with conventional methods and devices.

### SUMMARY OF THE INVENTION

Accordingly, it is an object of the present invention to overcome the above-mentioned disadvantages of the prior art by providing multiple-chamber devices that effect controlled delivery of fluids to a plurality of exposure zones in a target region of a substrate surface.

It is another object of the invention to provide a method for delivering cells to a plurality of exposure zones in a target region of a substrate surface.

It is a further object of the invention to provide a method for carrying out assays to detect cell-cell interactions at a plurality of exposure zones in a target region of a substrate surface.

Additional objects, advantages, and novel features of the invention will be set forth in part in the description that follows, and in part will become apparent to those skilled in the art upon examination of the following, or may be learned through routine experimentation upon practice of the invention.

In one embodiment, a device is provided for exposing a substrate surface to one or more fluids. The device comprises a substrate having a surface containing a contiguous target region and a cover plate having a surface capable of fluid-tight contact with the substrate surface. A plurality of fluid-transporting features is present in the cover plate surface, and the features are separated by at least one partitioning wall representing an integral portion of the cover plate. Each of a plurality of inlets is provided fluid communication with a fluid-transporting feature, and at least one outlet is associated with the plurality of fluid-transporting features. A means is provided for positioning the cover plate surface in fluid-tight contact with the substrate surface such that the at least one partitioning wall contacts a location in the contiguous target region. As a result, each fluid-transporting feature, in combination with the substrate surface, forms a flow passage containing a distinct exposure zone on the target region. Each distinct exposure zone is downstream from the inlet in fluid communication therewith and upstream from the at least one outlet. Optionally, The positioning means allows for the repositioning of the at least one partitioning wall to contact the contiguous target region at a different location.

The inventive device may be employed to carry out a method for exposing a substrate surface to plurality of fluids. The method involves providing a substrate and a cover plate as described above. The cover plate surface and the substrate surface are positioned in fluid-tight contact such that the at least one partitioning wall contacts the contiguous target region at a first location and that each fluid-transporting feature, in combination with the substrate surface, forms a flow passage containing a distinct exposure zone on the target region such that each distinct exposure zone is downstream from the inlet in fluid communication therewith and upstream from the at least one outlet. The method further involves maintaining one or more fluids in laminar flow from one or more sources through the inlets over the target region such that the one or more fluids contact the exposure zones on the target region. Once the exposure zones have been contacted by the one or more fluids, at least one additional fluid is maintained in contiguous laminar flow over the target region, wherein the at least one additional fluid contacts one or more secondary exposure zones on the target region that are different from the distinct exposure zones formed previously, thereby exposing the one or more secondary exposure zones to the at least one additional fluid.

In another embodiment, the invention provides a method for exposing a substrate surface to a plurality of cells. The method involves the use of a substrate having a surface containing a contiguous target region. Each of a plurality of fluids is maintained in contiguous laminar flow over the target region, wherein each fluid conveys a cell over a distinct exposure zone on the target region, thereby exposing the distinct exposure zone to the cell. Typically, the distinct exposure zones are defined at least in part by at least one partitioning wall contacting the contiguous target region.

In a further embodiment, the invention provides a method for detecting cell-cell interactions. As above, the method involves the use of a substrate having a surface containing a contiguous target region. A plurality of cells is immobilized in the contiguous target region, and at least one partitioning wall is placed in contact with the contiguous target region, thereby defining a plurality of distinct exposure zones on the target region. Each of a plurality of fluids is maintained in contiguous laminar flow over the target region. As a result, each fluid conveys a cell over a distinct exposure zone, thereby exposing any immobilized cells in the distinct exposure zone to the cell conveyed by the fluid. The method also involves detecting a cell-cell interaction, if present, in any of the distinct exposure zones as a result of the contact or proximity between a cell conveyed by a fluid and an immobilized cell.

### BRIEF DESCRIPTION OF THE DRAWINGS

FIGS. 1A-1B, collectively referred to as FIG. 1, illustrate an embodiment of the inventive device for exposing eight exposure zones of target region of a substrate surface to a fluid. FIG. 1A illustrates the device in exploded view. FIG. 1B illustrates the device in schematic top through view, before a stream containing a reagent is introduced therein.

FIGS. 2A and 2B, collectively referred to as FIG. 2, illustrate an embodiment of the inventive device for exposing two exposure zones of a target region of a substrate surface to a fluid. FIG. 2A illustrates the device in exploded view. FIG. 2B illustrates the device in schematic top through view.

FIGS. 3A-3D, collectively referred to as FIG.3, illustrate a method for using the device of FIG. 2 to carry out a cell-cell assay, wherein a monolayer of cells is immobilized over substantially the entire target region.

FIGS. 4A-4D, collectively referred to as FIG. 4, illustrate a method for using the device of FIG. 2, wherein cells are immobilized as an array through the use of a stencil over the target region.

FIGS. 5A and 5B, collectively referred to as FIG. 5, illustrate various arrays that can be used with the device of FIG. 2.

FIGS. 6A-6B, collectively referred to as FIG. 6, illustrate an embodiment of the inventive device that allow for the repositioning of the cover plate with respect to the substrate for sequentially exposing different exposure zones of a target region of a substrate surface to a plurality of fluids.

### DETAILED DESCRIPTION OF THE INVENTION

Before the invention is described in detail, it is to be understood that, unless otherwise indicated, this invention is not limited to particular materials, components, or manufacturing processes, as such may vary. It is also to be understood that the terminology used herein is for purposes of describing particular embodiments only, and is not intended to be limiting.

It must be noted that, as used in the specification and the appended claims, the singular forms “a,” “an,” and “the” include plural referents unless the context clearly dictates otherwise. Thus, for example, reference to “a lane” includes a single lane as well as a plurality of lanes, reference to “a reagent” includes a single reagent as well as a combination or mixture of reagents, reference to “an inlet” includes a single inlet as well as two or more inlets, and the like.

In this specification and in the claims that follow, reference will be made to a number of terms that shall be defined to have the following meanings:

The term “array” as used herein refers to a two-dimensional arrangement of features, such as cells, molecular moieties or exposure zones on a substrate surface. Arrays are generally comprised of regular features that are ordered, as in, for example, a rectilinear grid, parallel stripes, spirals, lanes, and the like; but non-ordered arrays may be advantageously used as well. An array differs from a pattern in that patterns do not necessarily contain regular and ordered features.

The term “concentration” as used herein refers to the ratio of the molar amount of a substance to fluid volume in a stream. The substance may be entirely soluble, partially soluble, or insoluble in the fluid of the stream.

The term “cell line” as used herein refers to a permanently established cell culture that will proliferate indefinitely if given appropriate fresh medium and space. While cell lines are readily available for some species, such as those in the rodent family, and difficult to establish for other species, such as humans, the term “cell line” as used herein is not limited to any particular species or cell type.

The term “expose” as to “expose an exposure zone of a substrate surface to a cell” is used in its ordinary sense and refers to subjecting an item, e.g., an exposure zone of a substrate surface, or allowing the item to be subjected to, the influence of another item, e.g., a cell, preferably via contact but optionally through mere proximity. The items “exposed” to each other may or may not interact.

The term “fluid-tight” is used herein to describe the spatial relationship between two solid surfaces in physical contact, such that fluid is prevented from flowing into the interface between the surfaces.

The term “fluid-transporting feature” as used herein refers to an arrangement of solid bodies or portions thereof that direct fluid flow. Fluid-transporting features include, but are not limited to, chambers, reservoirs, conduits, and channels. The term “conduit” as used herein refers to a three-dimensional enclosure formed by one or more walls and having an inlet opening and an outlet opening through which fluid may be transported. The term “channel” is used herein to refer to an open groove or a trench in a surface. A channel in combination with a solid piece over the channel forms a “conduit”.

The term “gradient,” as in “concentration gradient” or “chemical gradient,” is used herein in its ordinary sense and refers to the variation of a parameter, e.g., concentration, over a given distance. Gradients may be formed from simple or complex chemical structures. For example, entities that may form a gradient include, but are not limited to, biological entities such as proteins, peptides, antibodies, cells, viral particles, sugars, proteoglycans, and lipids.

The terms “immobilize,” “immobilized,” and “immobilizing,” e.g., as in “immobilized cells,” are used herein to describe the fixation of a cell to a position on a substrate surface such that movement of the cell does not occur as a result of mechanical forces applied to the cell solely as the result of fluid flow. For example, an immobilized cell exposed to a cellular suspension in laminar flow may not move in response to the fluid flow but may move as a result an interaction with a cell in the cellular suspension. Similarly, an immobilized cell exposed to a laminar flow that exhibits a chemical gradient may not move in response to the fluid flow but may exhibit chemotactic behavior and move in response to the chemical gradient.

The term “laminar flow” as used herein refers to fluid movement in the absence of turbulence, such that mixing of fluid components occurs solely or primarily as a result of diffusion. The Reynolds number associated with laminar flow as described herein is typically about 0.01 to about 200, preferably about 0.01 to 20, and optimally about 0.1 to 20.

The term “lane” as used herein refers to one of a set of typical routes or courses along which a fluid travels or moves. While a lane may be bounded by one or more solid surfaces, a lane of fluid is bounded by at least another fluid, with which nondiffusional mixing does not occur. Thus, a reagent in one lane of fluid bounded by another lane may diffuse across the boundary between the lanes.

“Optional” or “optionally” as used herein means that the subsequently described feature or structure may or may not be present, or that the subsequently described event or circumstance may or may not occur, or that the description includes instances where a particular feature or structure is present and instances where the feature or structure is absent, or instances where the event or circumstance occurs and instances where it does not.

The term “primary cell” is used herein in its ordinary sense and refers to a cell taken directly from a living tissue that has not been immortalized. Primary cells may be derived from a number of sources such as from an in vivo or ex vivo organ culture. For example, primary cells may be taken from a liver biopsy, a fetus, or embryonic tissue.

The term “reagent” is used herein to refer to any substance that is used in a chemical, biochemical, or biological reaction to detect, measure, examine, or produce other substances. Reagents may be contained in a fluid in solvated, partially solvated, or suspended form.

The term “substrate” as used herein refers to any material having a surface over which laminar fluid flow may occur. The substrate may be constructed in any of a number of forms such as wafers, slides, well plates, and membranes. Suitable substrate materials include, but are not limited to, supports that are typically used for cell handling, e.g.: polymeric materials (e.g., polystyrene, polyvinyl acetate, polyvinyl chloride, polyvinyl fluoride, polyacrylonitrile, polyacrylamide, polymethyl methacrylate, polytetrafluoroethylene, polyethylene, polypropylene, polybutylene, polyvinylidene fluoride, polycarbonate, polyimide, and polyethylene terephthalate); silica and silica-based materials; functionalized glasses; ceramics; and such substrates treated with surface coatings, polymeric, and/or metallic compounds, or the like. While the foregoing support materials are representative of conventionally used substrates, it is to be understood that the substrate may in fact comprise any biological, nonbiological, organic, and/or inorganic material, and may further have any desired shape, such as a disc, square, sphere, circle, etc. The substrate surface is typically but not necessarily flat, e.g., the surface may contain raised or depressed regions.

The term “surface modification” as used herein refers to the chemical, biological, and/or physical alteration of a surface by an additive or subtractive process to change one or more chemical and/or physical properties of a substrate surface or a selected location or region of a substrate surface. For example, surface modification may involve: (1) changing the wetting properties of a surface; (2) functionalizing a surface, i.e., providing, modifying, or substituting surface functional groups; (3) defunctionalizing a surface, i.e., removing surface functional groups; (4) otherwise altering the chemical composition of a surface, e.g., through etching; (5) increasing or decreasing surface roughness; (6) providing a coating on a surface, e.g., a coating that exhibits wetting properties that are different from the wetting properties of the surface; and/or (7) depositing particulates on a surface. Thus, for example, surface modification may involve providing a biologically derived coating on a surface, wherein the coating comprises a naturally occurring polymer such as a protein or peptide (e.g., collagen, fibronectin, albumin, fibrinogen, or thrombin), a saccharide (e.g., polymannuronic acid, polygalacturonic acid, dextran, or glycoaminoglycan), or a synthetic polymer (e.g., polyvinyl alcohol, acrylic acid polymers, and acrylic acid copolymers).

The term “target region” as used herein refers to a predefined two-dimensional area over which fluid is directed to flow. The target region is typically, but not necessarily, contiguous and may or may not have cells adhered thereto. In some instances, fluid may be directed to flow over the entirely of the target region. In other instances, fluid may be directed to flow over only portions of the target region, e.g., “exposure zones.” The target region may exhibit any of a variety of surface properties as long as the surface properties are predetermined. In some instances, for example, the target region may be functionalized so as to have surface reaction sites that allow a reagent to be attached thereto. In other instances, the target region may be selected for its ability to repel certain reagents.

Thus, the invention provides a device for exposing a substrate surface to one or more fluids. The device offers a convenient and efficient means to selectively expose portions of a substrate surface. The device comprises a substrate having a surface containing a contiguous target region and a cover plate having a surface capable of fluid-tight contact with the substrate surface. A plurality of fluid-transporting features is present in the cover plate surface, and the features are separated by at least one partitioning wall representing an integral portion of the cover plate. Each of a plurality of inlets is provided fluid communication with a fluid-transporting feature, and at least one outlet is associated with the plurality of fluid-transporting features. A positioning means is provided to position the cover plate surface in fluid-tight contact with the substrate surface such that the at least one partitioning wall contacts a location in within the contiguous target region. As a result, each fluid-transporting feature, in combination with the substrate surface, forms a flow passage containing a distinct exposure zone on the target region. Each distinct exposure zone is downstream from the inlet in fluid communication therewith and upstream from the at least one outlet. The contact between the at least one partitioning wall and the contiguous target region represents an improvement over known fluid delivery devices in a number of ways as discussed below.

Typically, the substrate is detachable from the cover plate, and the substrate surface is substantially planar. In addition, the fluid-transporting features are preferably substantially identical parallel channels that each defines a flow direction from an upstream to a downstream terminus, and the flow directions of the channels are the same. In most instances, the channels each have a width of about 0.1 to about 500 micrometers. Preferably, the channels each have a width of about 200 to about 400 micrometers. Typically, the one or more partitioning walls should have a width smaller than that of the channels. Optimally, the width of the partitioning wall should be minimized but should not be so narrow as to compromise the performance of the device. Since fluid tight contact is desired between the cover plate and substrate surfaces, the width of the partitioning wall should impart sufficient strength and rigidity to enable fluid-tight contact between the cover plate surface associated with partitioning wall and the target region. In addition, one or more sources of fluids may be provided, depending on the desired use of the device. As a rule, each inlet is in fluid communication with a source of fluid. In some instances, each inlet is in fluid communication with a different source of fluid. In other instances, each inlet is in fluid communication with the same source of fluid.

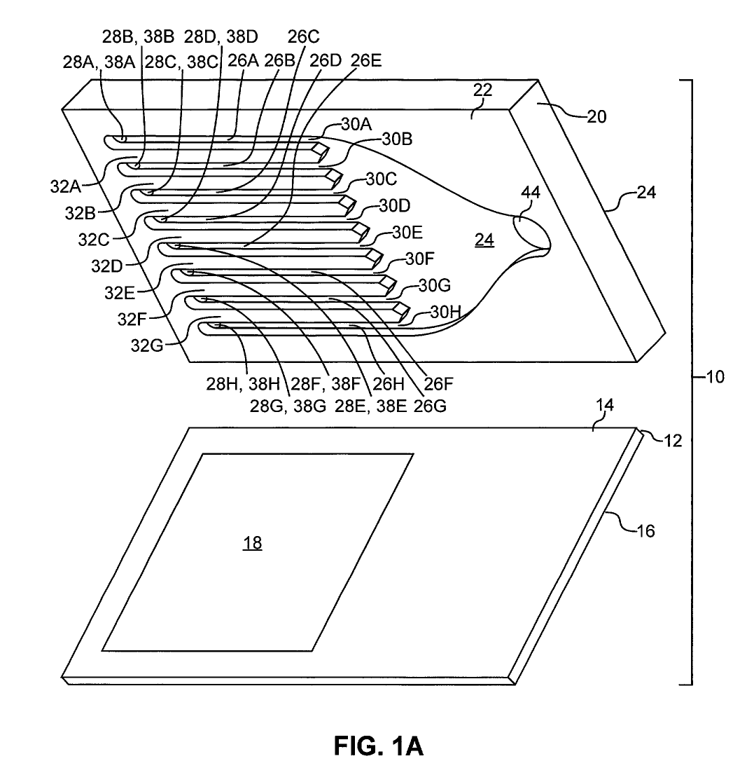

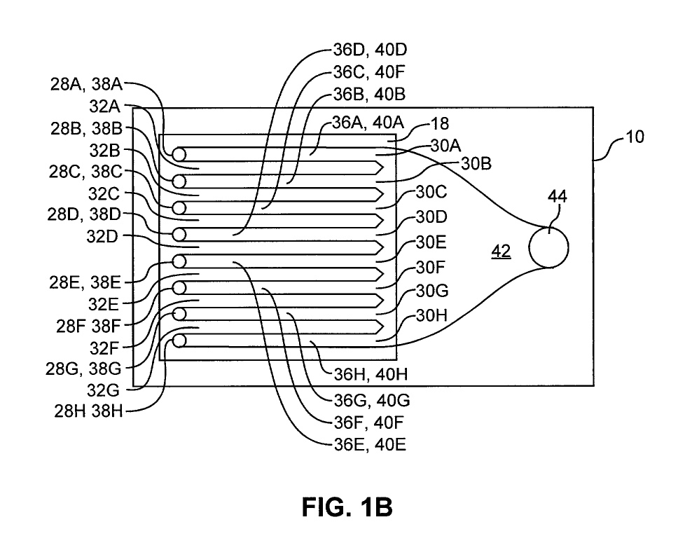

FIG. 1 illustrates an embodiment of the inventive device. As with all figures referenced herein, in which like parts are referenced by like numerals, FIG. 1 is not necessarily to scale, and certain dimensions may be exaggerated for clarity of presentation. The device 10 includes a substrate 12 comprising first and second substantially planar opposing surfaces indicated at 14 and 16, respectively, and is comprised of a material that is substantially inert with respect to the fluids that will be transported through the device. The surfaces 14 and 16 are rectangular in shape and parallel to each other. While FIG. 1 illustrates that a square-shaped target region 18 is located at the center of surface 14, the target region may be of any size (or shape) as long as it is no larger than surface 14. For square-shaped target regions, the surface area of the target region is typically 1 mm² to about 100 mm², preferably about 10 mm² to about 50 mm², and optimally about 20 mm² to about 30 mm².

The device 10 also includes a cover plate 20 having first and second substantially planar opposing surfaces indicated at 22 and 24, respectively. The contact surface 22 of the cover plate 20 is typically capable of interfacing closely with the contact surface 14 of the substrate 12 to achieve fluid-tight contact between the surfaces. Eight identical elongate channels, indicated at 26A-26H, are located on the first surface 22 of the cover plate 20. Each of the channels 26A-26H extends parallel to the other channels from an associated upstream terminus 28A-28H toward a downstream terminus 30A-30H. The length of each channel is equal to the length of a side of the target region 18. Seven partitioning walls, indicated at 32A-32G, separate the channels. All of the downstream termini fluidly communicate with a collection fluid-transporting feature 34.

The cover plate 20 may be substantially immobilized over, and aligned with, the substrate contact surface 14 such that the location of the target region 18 coincides with the location the channels 26A-26H. That is, the target region and the channels are superimposed over each other. As a result, illustrated in FIG. 1B, the target region 18 of the substrate contact surface 14 in combination with each of the channels 26A-26H defines flow passages in the form of conduits 36A-36H through which fluids may flow. Each conduit 36A-36H is provided fluid communication with an inlet. While the inlets may be constructed in any of a number of different ways, FIG. 1A illustrates that the inlets are provided in the form of conduits, indicated at 38A-38H, that extend though opposing surfaces 22 and 24 of the cover plate 20. Portions of the target region that form interior surfaces of the conduits serve as distinct exposure zones 40A-40H. Similarly, the substrate contact surface 14 in combination with the collection fluid-transporting feature 34 forms a collection fluid-transporting passage 42 downstream from the conduits 36A-36H. In addition, a single outlet 44 in the form of a conduit extends through opposing surfaces 22 and 24 of the cover plate 20. Thus, the single outlet 44 fluidly communicates with conduits 36A-36H located upstream therefrom via collection fluid-transporting passage 42.

Thus, as illustrated in FIG. 1B, the device is assembled such that one or more fluids from inlets 38A-38H flow through conduits 36A-36H. As a result, distinct exposure zones 40A-40H are each exposed to the fluid flowing through the associated conduit. Fluids having flowed through the flow passages are collected by the collection fluid-transporting passage 42 and leave the device through outlet 44.

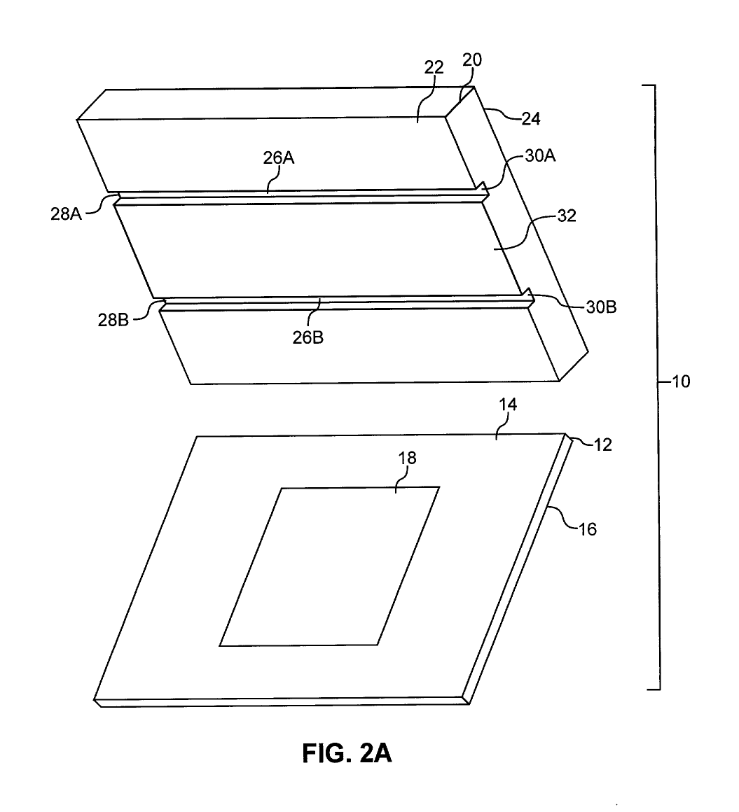

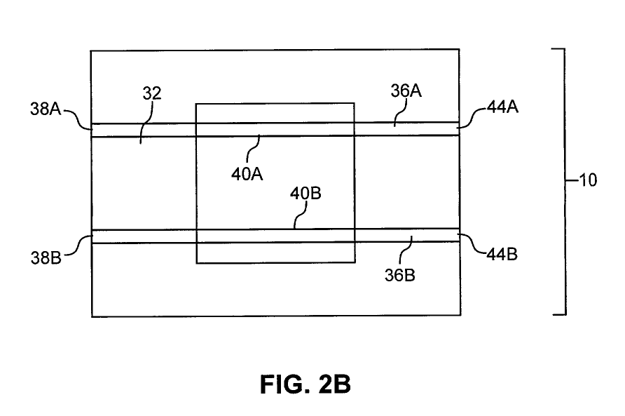

FIG. 2 illustrates another embodiment of the inventive device. This device is similar to the device illustrated in FIG. 1 in that a substrate 12 is also provided comprising first and second substantially planar opposing surfaces indicated at 14 and 16, and that a square-shaped target region 18 is located at the center of surface 14. In addition, the device 10 also includes a cover plate 20 having first and second substantially planar opposing surfaces indicated at 22 and 24, respectively. As shown, the cover plate surface 22 is approximately the same size and shape as the substrate surface 14. In this embodiment, two channels, indicated at 26A-26B, are located on the first surface 22 of the cover plate 20. The channels 26A and 26B extend parallel to each other along the length of the cover plate 20. Thus, both upstream termini 28A and 28B and downstream termini 30A and 30B are located at the opposing edges of surface 22, and the length of the channels is equal to the length of the cover plate. A partitioning wall 32 separates the channels.

The cover plate 20 may be substantially immobilized over, and aligned with, the substrate contact surface 14 such that the target region 18 and at least a portion of channels 26A and 26B are superimposed over each other. As a result, illustrated in FIG. 2B, the target region 18 of the substrate contact surface 14 in combination with each of the channels 26A and 26B defines conduits 36A and 36B through which fluids may flow. In addition, inlets 38A and 38B as well as outlets 44A and 44B are formed at the upstream and downstream termini of the conduits 36A and 36B, respectively. Again, portions of the target region that form interior surfaces of the conduits serve as distinct exposure zones 40A and 40B. When the device is assembled, one or more fluids from inlets 38A and 38B flow through conduits 36A and 36B and leave the device through outlets 44A and 44B. As a result, distinct exposure zones 40A and 40B are each exposed to the fluid flowing through the associated conduit.

In any of the above embodiments, suitable materials for forming the substrates and cover plates are selected with regard to physical and chemical characteristics that are desirable for proper functioning of the device. Suitable materials for forming the present devices include, but are not limited to, polymeric materials, ceramics (including aluminum oxide and the like), glass, metals, composites, and laminates thereof. The devices of the invention may also be fabricated from a “composite,” i.e., a composition comprised of unlike materials.

In addition, it is preferred that the device be constructed in a modular manner to ensure interchangeability of the components. In particular, stock items can be used to form certain components, thereby lowering the overall cost of the device and rendering it feasible, if desired, to dispose of the stock items after use. For example, the substrate may consist of an ordinary 25 mm×75 mm or 50 mm×75 mm glass slide, an item found in most laboratories. Similarly, to facilitate handling, the components of the inventive device may be detachable from one another. As access to the target region of the substrate is limited when it is in an opposing relationship to the cover plate, it is preferred that the substrate be detachable from the cover plate. Using a detachable and disposable item as the substrate, such as a glass slide, avoids the complex capillary tube attachment procedures before each use of the device that are required when tubes are essentially permanently connected to the inlets.

Thus, the cover plate and the substrate may each be discrete components. In such a case, alignment means such as a plurality of appropriately arranged protrusions in component parts, e.g., projections, depressions, grooves, ridges, guides, or the like, known to one of ordinary skill in the art, may be employed to align the cover plate with the base. In some instances, however, the substrate and the cover plate may be attached to each other. For example, the cover plate and the base may be hinged together to provide repeatable contact between the contact surfaces thereof. In such a case, the hinge also serves as an alignment means.

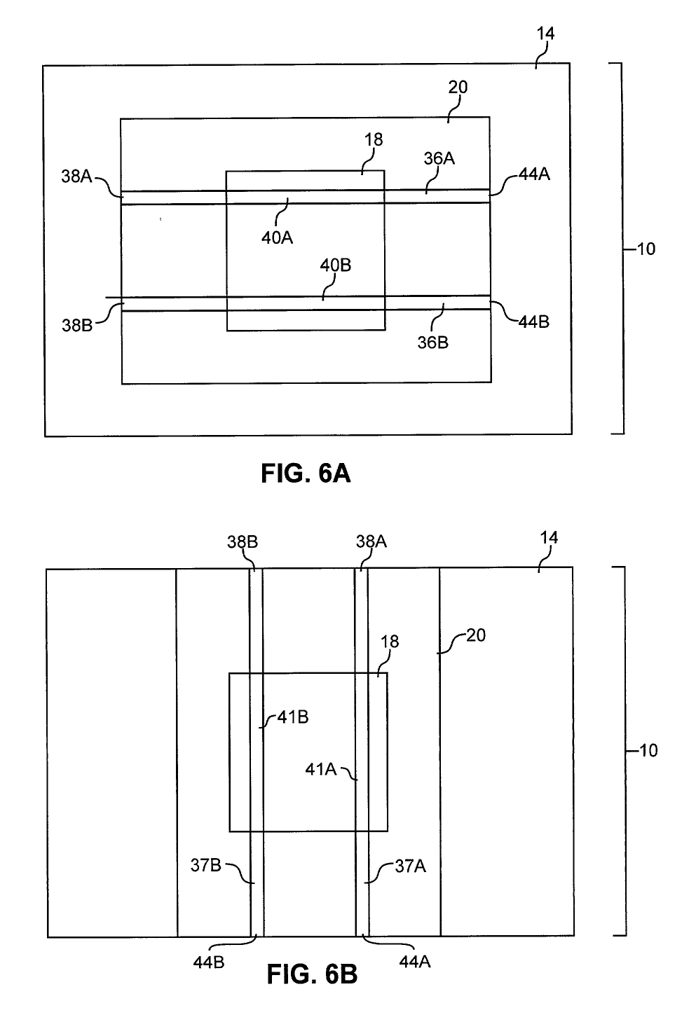

When the contact surfaces of the cover plate and the substrate are in fluid-tight contact, each flow passage is fluid-tight as well. To ensure that the flow passage is fluid-tight, pressure-sealing techniques may be employed, e.g., by using positioning means external or otherwise to urge the pieces together (such as clips, tension springs, or an associated clamp). Additionally or alternatively, the positioning means may hold the base and the cover plate together through appropriate application of a vacuum. As with all embodiments described herein, the sealing techniques may allow the contact surfaces of the cover plate and the base to remain in fluid-tight contact under a pressure associated with laminar fluid flow, i.e., an internal device fluid pressure of up about 5 bars, typically about 2 bars to about 5 bars, optimally about 2 bars. It is noted, however, that fluid-tight contact between the substrate and the cover plate may, but does not necessarily, involve direct contact. In some instances, positioning means allows for the repositioning of the at least one partitioning wall to contact the contiguous target region at a different location. The repositioning means may allow for the rotational reorientation of the cover plate and the substrate surfaces. This is illustrated in FIG. 6 and discussed below.

The inventive device may be constructed to deliver any number of fluids with or without reagents to the exposure zones. Commercially available fluid handling apparatuses, e.g., autosamplers and microtiter plates, may handle a fixed number of fluids, and the inventive device may be constructed to interface with these apparatuses. As such, apparatuses are ordinarily constructed to handle 8, 96, 384, or 1536 different fluids. Thus, the device may include a corresponding number of inlets and/or exposure zones as well. The one or more fluids may be aqueous and/or nonaqueous. Nonaqueous fluids include, for example, organic solvents and lipidic liquids. When the invention is employed to carry out cellular assays, as described below, typical reagents include but are not limited to, pharmacologically active agents and stains.

The specific geometry of the device components may vary depending on the intended use of the device. For example, one or more fluids may be maintained in contiguous laminar flow at a constant volumetric flow rate and velocity over the exposure zones and through the outlet. Whether fluid flow is laminar depends on several variables, such as: the geometry of the surfaces over which the fluid flows, flow velocity, and fluid properties such as viscosity. It is thus important that fluid movement in the inventive device be precisely controlled to maintain laminar flow. As components of this control, inlets typically have a cross sectional area of 1×10⁻⁵ mm² to about 1 mm², preferably about 5×10⁻⁴ to about 0.1 mm², and optimally 1×10⁻³ mm² to about 1×10⁻² mm². The inlets may have a variety of shapes including, but not limited to, circular, oval, square, rectangular, and triangular. Similarly, the shape of fluid-transporting features associated with the exposure zones may vary as well. For example, the fluid-transporting features may be hemicylindical, hemispherical, cubic, or tetrahedral. Accordingly, the exposure zone channels may be rectangular, circular, square, or triangular. Preferably, the exposure zones are identical in shape and size. In addition, although the channels have been represented in a generally extended form, channels for this and other embodiments can have a variety of configurations, such as a straight, serpentine, spiral, or any tortuous path. Further, the channels can be formed in a wide variety of channel geometries, including semi-circular, rectangular, rhomboidal, and the like; and the channels can be formed in a wide range of aspect ratios. When channels are provided, it is preferred that the channels are parallel and allow fluid to flow in the same direction.

Constant flow rate may be achieved through a number of means known in the art. In order to ensure that laminar flow is exhibited in the lanes formed downstream from the carrier liquid, a pump is employed to deliver appropriate fluid from a fluid source through the appropriate inlet. Typically, high precision microsyringe pumps are employed to provide fluid flow through capillaries to the inlets. Other types of pumps, however, may be employed. In some instances, one pump is sufficient to provide a motive force to ensure proper fluid flow. That is, each inlet may fluidly communicate with a source of reagent that is pressurized by the same pressure generating means. In other instances, however, each inlet may fluidly communicate with an independently controlled pressure generating means. While independent control of fluid introduction into the flow path typically involves added cost, such control allows for serial formation of lanes. Thus, selected portions of the target region may be exposed to reagents for differing periods. For example, if each of a plurality of inlets is adapted to allow through transport of the same reagent-containing fluid, independent control allows different portions of the target region to be exposed to the same reagent for different periods. This allows for the systematic study of the effect of a reagent on a target region as a function of time.

In another embodiment, the invention provides a method for exposing a substrate surface to a plurality of cells. The method involves the use of substrate having a surface containing a contiguous target region. Each of a plurality of fluids is maintained in contiguous laminar flow over the target region, wherein each fluid conveys a cell over a distinct exposure zone on the target region, thereby exposing the distinct exposure zone to the cell. When the method is practiced to carry out an assay involving cell-cell interaction, one or more cells may be immobilized in at least one of the distinct exposure zones. The method may involve detecting a cell-cell interaction, if present, in any of the distinct exposure zones as a result of the contact or proximity between a cell conveyed by a fluid and an immobilized cell.

The inventive method may be practiced using the inventive device as described above. Thus, the distinct exposure zones may be defined at least in part by at least one partitioning wall in contact with the contiguous target region. However, other devices may be employed to carry out the inventive method as well. The inventive method can be used with a cytometric device suitable for delivery of cells to different exposure zones, simultaneously or in series. For example, U.S. patent application Ser. No. 09/941,944 describes devices that allow the formation of one or more fluid lanes on a substrate surface, to expose a portion of a target region on the surface to one or more lanes of fluids. The fluid lanes may be employed to convey cells over distinct exposure zones on the target region, thereby exposing the distinct exposure zone to the cell. Similarly, hydrodynamic focused flow, e.g., as described in U.S. patent application Ser. No. 09/896,484, may be used to convey cells over distinct exposure zones on the target region.

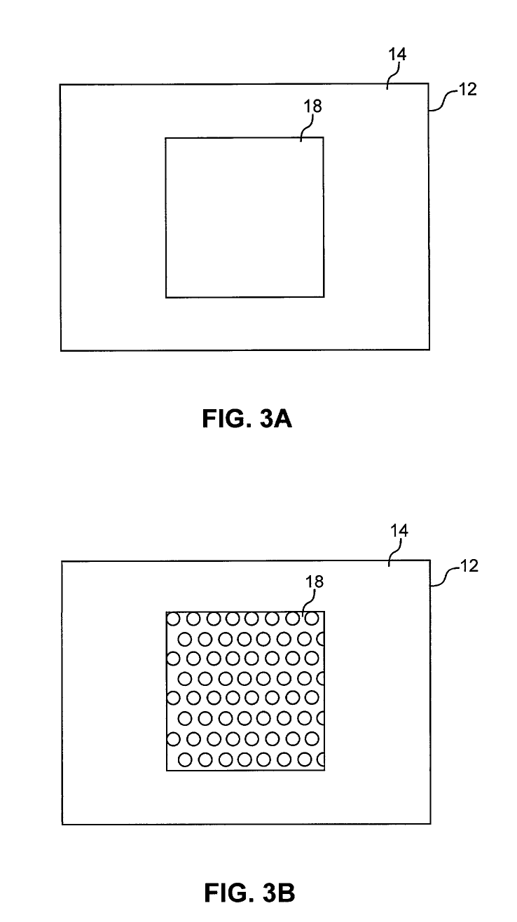

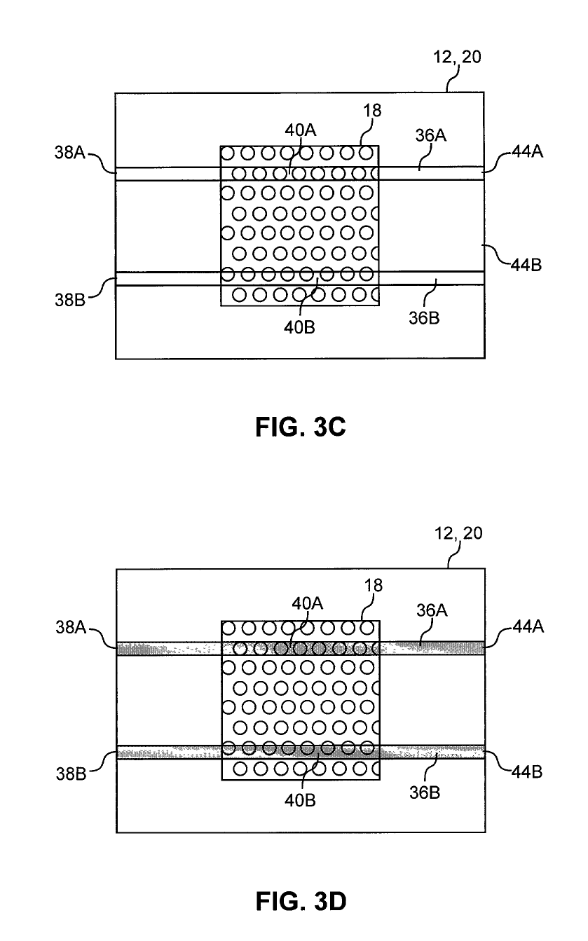

FIG. 3 illustrates a method for using the device of FIG. 2. In FIG. 3A, the device of FIG. 2 is provided in an unassembled form such that the substrate 12 is provided as a separate item from the cover plate. In FIG. 3B, the entire target region is coated with a cell-adhering agent, and a layer of cells is placed in contact with the cell-adhering agent. The cell layer is thereby immobilized over substantially the entire target region 18. As discussed below, cell adhesion may be accomplished through use of any of a number of known means. As shown, the layer of cells covers the entire target region 18 but nowhere else on the substrate surface 14. Although this is preferred, coverage of no more than the target region by the layer of cells is not required. The cover plate 20 then may be substantially immobilized over, and aligned with, the substrate contact surface 14 in the manner depicted in FIG. 3C, such that the target region 18 and the channels 26A and 26B are superimposed over each other. As a result, the target region 18 in combination with each of the channels 26A and 26B defines fluid-tight conduits 36A and 36B through which fluids may flow. Furthermore, because the entire target region 18 is covered with a layer of cells, the entirety of exposure zones 40A and 40B has a layer of cells immobilized thereon. Inlets 38A and 38B are each provided fluid communication with a source of fluid that contains cells.

Thus, as depicted in FIG. 3D, the cells located in exposure zones 40A and 40B are each exposed to the fluid flowing through conduits 36A and 36B, respectively, and to the cells conveyed by the fluids. When fluid-tight contact is achieve between the cover plate surface 22 and the substrate surface 14, the cells interposed between the cover plate surface 22 associated with the partitioning wall 32 and the target region 18 are isolated from the fluids flowing through conduits 36A and 36B. As shown, the cells are conveyed in single file over each exposure zone, though this is not a necessity. Fluids having flowed through the flow passages leave the device through outlets 44A and 44B, respectively. Depending on whether the cells conveyed by the fluids over the immobilized cells interact with the immobilized cells, the cells may be conveyed through outlets 44A and 44B as well. Cell-cell interactions may be detected within conduits 36A and 36B and/or deduced by examining the cells leaving the device through outlets 44A and 44B.

Thus, FIG. 3 also illustrates an example of a method for detecting cell-cell interactions that provides a number of advantages over known methods for detecting cell-cell interactions. One advantage is the simplicity of the method. Unlike methods that allow for the detection of different yet simultaneous cell-cell interactions, this method does not require the use of a cellular array. This is advantageous because it eliminates the need for the time and effort associated with the production of cellular arrays. In addition, the method is robust because precise alignment between the fluid delivery mechanism and the immobilized cells is not required. That is, depending on the construction of the fluid delivery device, the method can be successfully carried out even if the cover plate and the substrate are slightly misaligned. This, of course, means that precision positioning means are neither needed nor precluded for use with in the present invention.

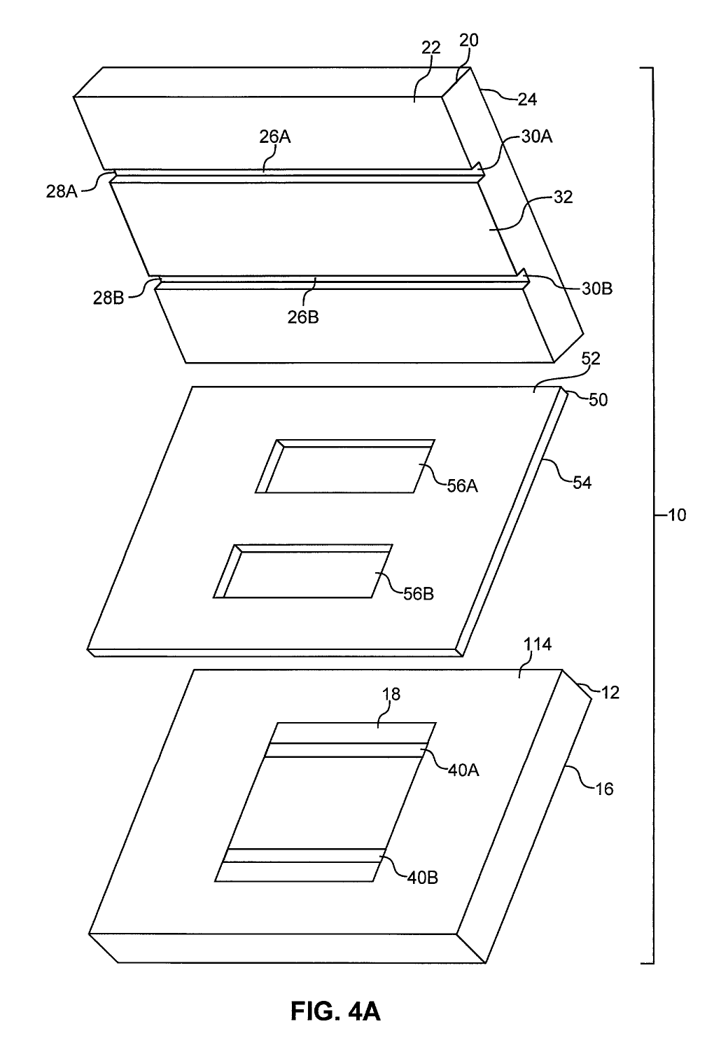

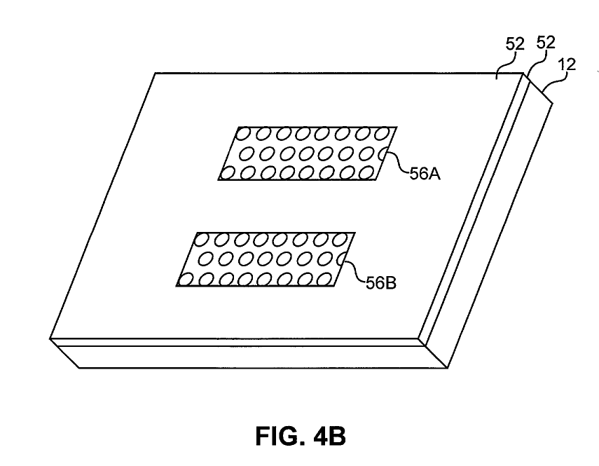

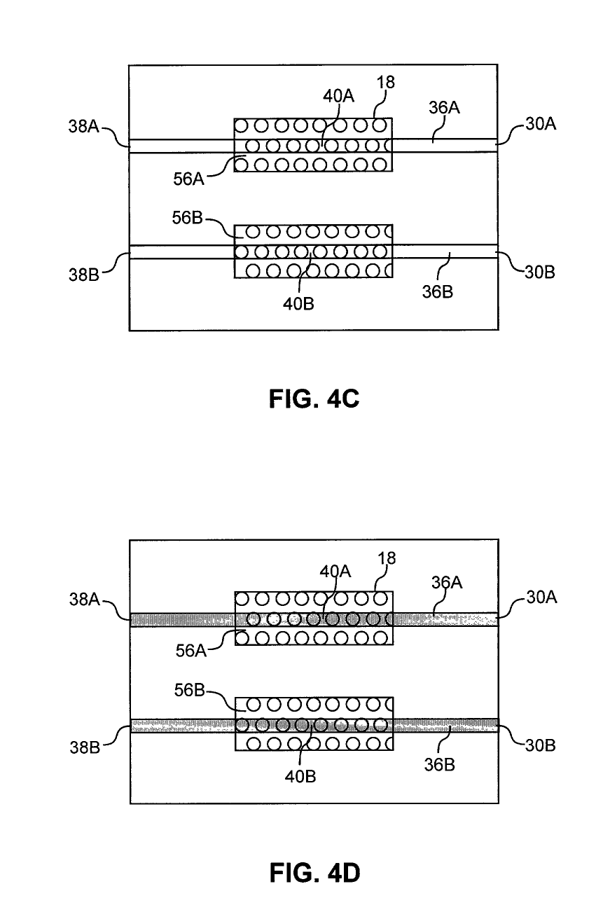

The device may be used in conjunction with array technology as well. FIG. 4 illustrates a method similar to that illustrated in FIG. 3, except that cells are immobilized as an array over the target region. As depicted in FIG. 4A, the device of FIG. 2 is provided in an unassembled form such that the substrate 12 and cover plate 20 are separate items. In addition, a stencil is provided in the form of a plate 50 having parallel surfaces indicated at 52 and 54, and feature holes 56A and 56B extending though the plate 50 from surface 52 to surface 54. The size, shape, and location of the feature holes 56A and 56B are selected according to the exposure zones 40A and 40B, respectively, on the target region 18. However, the feature holes and associated exposure zones are preferably, but do not have to be, identical in size or shape.

As illustrated in FIG. 4B, a plurality of cells is immobilized on the target region 18 as cellular features of an array. This can be accomplished through any of a number of ways known in the art for patterning cells. Here, the cellular array is formed through the use of the stencil 50. The entire target region is first coated with a cell-adhering agent, and the stencil 50 is placed on the substrate 12 such that stencil surface 54 contacts that cell-adhering agent on the substrate surface 14. Then, feature holes 56A and 56B are positioned in target region 18 such that they coincide with the location of the exposure zones 40A and 40B, respectively. Alternatively, the stencil may be placed on the surface before the cell-adhering agent is selectively coated on the target region, thus using the stencil to mask the portions of the substrate surface that are not target zones. In either case, cells are placed in contact with the cell-adhering agent through the stencil and thus form cellular feature on the exposure zones.

The cover plate 20 then may be substantially immobilized over, and aligned with, the substrate contact surface 14 in the manner depicted in FIG. 4C, such that the target region 18 and the channels 26A and 26B are superimposed over each other. The stencil may or may not be removed before the cover plate is immobilized in place. As a result, the target region 18 in combination with each of the channels 26A and 26B defines fluid-tight conduits 36A and 36B through which fluids may flow. Furthermore, exposure zones 40A and 40B each has a cellular feature immobilized thereon. Inlets 38A and 38B are each provided fluid communication with a source of fluid that contains cells. Thus, as depicted in FIG. 4D, the cells located in exposure zones 40A and 40B are each exposed to the fluid flowing through conduits 36A and 36B, respectively, and to the cells conveyed by the fluids.

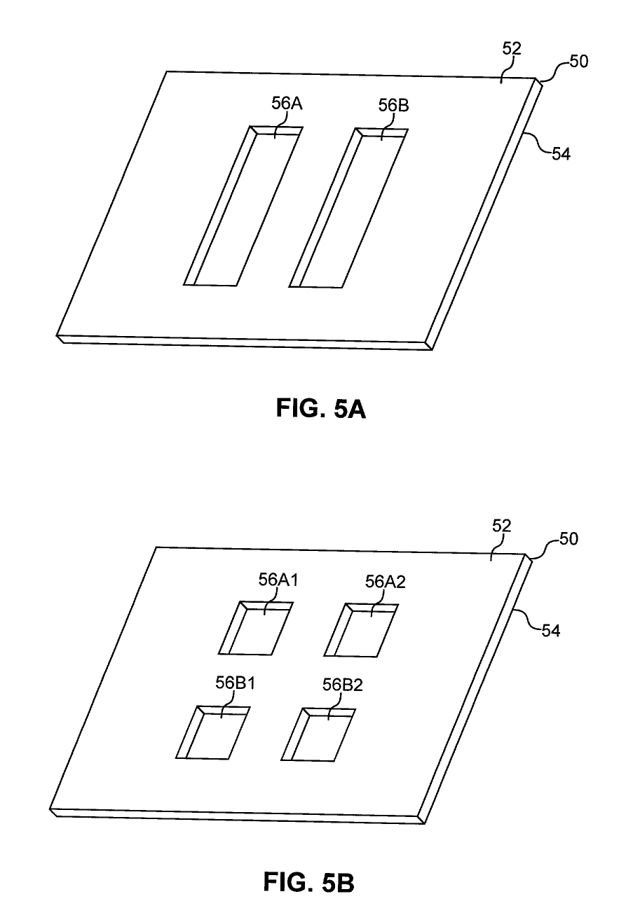

Thus, it is evident that arrays comprised of cellular or other features may be employed with the inventive method. FIG. 5 illustrates various arrays suitable for use with the device of FIG. 2. In FIG. 5A, the stencil depicted in FIG. 4 is used to form an array of features that is rotated ninety degrees from the array formed in FIG. 4. This array configuration allows the cells conveyed in each of conduits 36A and 36B to interact with two different features formed from feature holes 56A and 56B, sequentially. Similarly, FIG. 5B illustrates an array of features that allows cells conveyed in each of conduits 36A and 36B to interact with two different features in parallel. That is, conduit 36A allows cells conducted therein to interact with features formed from feature holes 56A1 and 56A2, and conduit 36B allows cells conducted therein to interact with features formed from feature holes 56B1 and 56B2. Thus, it is evident that features may be located in a single exposure zone or a plurality of exposure zones.

The inventive device may also be used to sequentially expose different exposure zones of the target region to a plurality of fluids. For example, after the one or more fluids contacts the exposure zones, at least one additional fluid is maintained in contiguous laminar flow over the target region. In such a case, the at least one additional fluid contacts one or more secondary exposure zones on the target region that are different from the distinct exposure zones formed previously. This may be carried out by repositioning the cover plate surface with respect to the substrate surface such that the at least one partitioning wall contacts the contiguous target region at a different location. The repositioning of the cover plate forms a plurality of secondary distinct exposure zone on the target region. The repositioning may involve rotationally reorienting the cover plate surface over the substrate surface. Typically, the rotational reorientation involves an angular rotation of about 60° to 120°. Preferably, the angular rotation is about 90°.

FIG. 6 illustrates a device that allows for the sequentially exposure of different exposure zones of the target region to a plurality of fluids. The device depicted in FIG. 6 is similar to that illustrated in FIG. 2, except that the width of the substrate is the same as the length of the cover plate. When the cover plate 20 is substantially immobilized over, and aligned lengthwise with the substrate contact surface 14, conduits 36A and 36B, as depicted in FIG. 6A, are formed through which fluids may flow. In addition, inlets 38A and 38B as well as outlets 44A and 44B are formed at the upstream and downstream termini of the conduits 36A and 36B, respectively. Again, portions of the target region that form interior surfaces of the conduits serve as distinct exposure zones 40A and 40B. When the device is assembled in this manner, one or more fluids from inlets 38A and 38B flow through conduits 36A and 36B and leave the device through outlets 44A and 44B. As a result, distinct exposure zones 40A and 40B are each exposed to the fluid flowing through the associated conduit.

In order to allow for subsequent exposure of different exposure zones on the target region, the device as illustrated in FIG. 6A is disassembled and reassembled in the manner depicted in FIG. 6B. As shown in FIG. 6B, the cover plate 20 is substantially immobilized over and aligned perpendicularly to the substrate contact surface 14. As a result, conduits 37A and 37B are formed through which fluids may flow. Again, inlets 38A and 38B as well as outlets 44A and 44B are formed at the upstream and downstream termini of the conduits 36A and 36B, respectively. Again, portions of the target region that form interior surfaces of the conduits serve as distinct secondary exposure zones 41A and 41B. When the device is assembled in this manner, one or more fluids from inlets 38A and 38B flow through conduits 37A and 37B and leave the device through outlets 44A and 44B. As a result, secondary distinct exposure zones 41A and 41B are each exposed to the fluid flowing through the associated conduit.

In summary, the invention provides a means for carrying out various chemical processes such as surface-modification methods as well as cell-based assays. In order to carry out such assays, at least one exposure zone contains at least one cell. Typically, each distinct exposure zone contains a cell. In some instances, each distinct exposure zone contains a different type of cell. In other instances, each distinct exposure zone contains the same type of cell. When each distinct exposure zone contains a plurality of cells, the cells may be the same or different. As discussed above, the entire target region may be covered with a plurality of cells. Alternatively, an array of cellular features is present on the target region. Preferably, the cells on the target region form a cellular monolayer immobilized on the target region by a cell-adhering substance. That is, the monolayer may be substantially contiguous or comprise an array of features, each feature comprising at least one cell.

In addition, the invention also may provide for fluid in laminar flow conveying one or more cells. Thus, at least one source of fluid may contain a suspension of cells. In some instances, the suspension contains cells of different types. In other instances, the suspension contains cells of the same type. In addition, at least one source of fluid may contain a body fluid. Thus, for example, a source of fluid may contain whole blood, a body fluid that contains cells of different types. In some instances, at least one source of fluid contains a candidate compound for interaction with a cell.

When live cells are used, either immobilized on the substrate or conveyed by the fluid flow, it is preferred that the fluid flowing over the exposure zones comprises a culture medium for sustaining cell viability. It must be noted, however, that the culture medium does not necessarily ensure that the cell remains living, although living cells are preferred. Culture media suitable for any particular cell will be known to those skilled in the art and are available commercially from, for example, Sigma Inc., St. Louis, Mo. Generally, such media contain mixtures of salts, amino acids, vitamins, nutrients, and other substances necessary to maintain cell health. Preferred salts in the culture medium include, without limitation, NaCl, KCl, NaH₂PO₄, NaHCO₃, CaCl₂, MgCl₂, and combinations thereof. Preferred amino acids are the naturally occurring L amino acids, particularly arginine, cysteine, glutamine, histidine, isoleucine, leucine, lysine, methionine, phenylalanine, threonine, tryptophan, tyrosine, valine, and combinations thereof. Preferred vitamins in the cell culture include, for example, biotin, choline, folate, nicotinamide, pantothenate, pyridoxal, thiamine, riboflavin, and combinations thereof. Glucose and/or serum, e.g., horse serum or calf serum, are also preferred components of the culture medium. Optionally, antibiotic agents such as penicillin and streptomycin may be added to suppress the growth of bacteria. Preferably, the culture medium will contain one or more protein growth factors specific to a particular cell type. For example, many nerve cells require trace amounts of nerve growth factor (NGF) to sustain their viability. Similarly, the culture medium will preferably contain hepatocyte growth factor (HGF) when hepatocytes are present in the assay. Those skilled in the art routinely consider these and other factors in determining a suitable culture medium for any given cell type. The culture medium can be present in one or both of the guide streams and optionally in the fluid stream containing the reagent.

Nearly any type of cell may be used with the invention, regardless of whether immobilized on the exposure zone or conveyed by a fluid over the exposure zone. For example, either or both eukaryotic cells and prokaryotic cells may be used. In some instances, primary cells obtained from a mammal, e.g., a human, are employed. Preferred cell types are selected from the group consisting of blood cells, stem cells, endothelial cells, epithelial cells, bone cells, liver cells, smooth muscle cells, striated muscle cells, cardiac muscle cells, gastrointestinal cells, nerve cells, and cancer cells. Alternatively, the employed cells may originate from a cell line.

The substrate surface on which the target region is located may be selected for facile immobilization of cells. Such solid surfaces include, for example, a collagen-derivatized surface, dextran, polyacrylamide, nylon, polystyrene, and combinations thereof. In some instances, the solid surfaces are formed through surface modification techniques. Regardless whether the surfaces are modified or inherently cytophilic, the surfaces allow cells to be immobilized thereon using conventional techniques known to those skilled in the art. For example, the cells may be immobilized on the target region by simply contacting the target region with the cells. Optionally, a centrifuge may be used. Generally, the force required to immobilize a cell on the target region is from about 200×g to about 500×g.

Alternatively, the surface may be coated with a layer of a cell-adhering substance, such as collagen, alginate, agar, or other material to immobilize the cells. When immobilization of cells in a contiguous layer is desired, the cell-adhering substance may be contiguously coated on the target region. When it is desirable to provide an immobilized array of cells, however, the cell-adhering substance may be present as an array of features on the target region. That is, an array of locations on the target region may be coated with an appropriate material to form an array, e.g., patterns such as lanes, checkerboards, spots, or others, so that cells may be spatially arranged at specific locations on the solid surface. See, e.g., U.S. Pat. Nos. 5,976,826 and 5,776,748 to Singhvi.

Alternatively, the cells may be present on the target region as a tissue sample. Immobilization of tissue samples containing cells of interest may be accomplished by first freezing, e.g., to about −15° C. to about −20° C., a relatively large section of tissue. Thereafter, a knife, microtome, or similar sectioning device is used to slice the frozen tissue into sections. Next, a single section of the tissue is placed onto the target region, e.g., a glass slide, and the section is allowed to “melt” on the target region, thereby immobilizing the cells in the tissue on the target region. Those skilled in the art will recognize other immobilization techniques that can be used.

As stated above, the present method provides a method for screening the interaction between cells or biological activity of a cell with respect to a particular reagent. Biological activity of the reagent can be detected by determining whether the cell changes in response to the reagent, for example, by changing its shape, exhibiting chemotactic behavior, or expressing a protein. Generally, a means for visually observing or otherwise detecting such changes is used. Such means include, for example, use of a microscope, chromatographic methods, an immunoassay, a fluorescence detector, a radioactivity detector, and combinations thereof.

Thus, variations of the present invention will be apparent to those of ordinary skill in the art. In addition, it is to be understood that while the invention has been described in conjunction with the preferred specific embodiments thereof, that the foregoing description and the following examples are intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

### EXAMPLES

The practice of the present invention will employ, unless otherwise indicated, conventional techniques of the analytical industry and the like, which are within the skill of the art. Such techniques are explained fully in the literature.

In the following examples, efforts are made to ensure accuracy with respect to numbers used (e.g., amounts, temperature, etc.) but some experimental error and deviation should be accounted for. Unless indicated otherwise, temperature is in degrees Celsius and pressure is at or near atmospheric. All reagents are obtained commercially unless otherwise indicated.

### Examples 1

The inventive device is used to evaluate the effect of each of seven different compounds on the binding interaction between primary human umbilical endothelial cells (HUVECs) with respect to T-cell clones (Jurkat cells). The evaluation begins when HUVECs are prepared by placing the cells on a target region of a slide having a contiguous target region over a surface that allows the HUVECs to adhere thereto. The cells are cultured in media to which a 10% fetal calf serum (FCS) solution is added. A sufficient amount of time is allowed to pass until the cells reached confluency such that a monolayer of HUVECs cover substantially the entire contiguous target region. HUVECs are then treated with tumor necrosis factor-alpha (TNF) at a concentration of 100 μg/ml for 24 hours.

On the following day, eight samples are prepared. Each sample 1 ml aliquot of Jurkat cells (T-cell clones) treated with Calcein-AM (1-4 μM). Then, different candidate compounds are introduced into seven of the eight samples. The eighth sample contains no candidate compound and serves as a control. The HUVEC-containing slide is placed over the stage heater adjusted to 37° C. to bring the slide to temperature. A cover plate as illustrated in FIG. 1 is placed in fluid-tight contact with the slide surface such that the partitioning walls contact the contiguous target region. Thus, eight flow passages are formed, each containing a distinct exposure zone having cells adhered thereto, wherein each distinct exposure zone is downstream from the inlet in fluid communication therewith and upstream from the outlet. Each flow passage is then purged with HBSS (Hank's balanced saline solution).

Samples of fluorescently labeled Jurkat cells are loaded into a microtiter well from which an autosampler is programmed to sample and pump fluid at 0.02 μl/sec simultaneously into each of the flow passages. Laminar flow is maintained at all times for fluids flowing through the flow passage The cells are loaded for one minute, followed by a no-loading period of 1-10 minutes to allow cell binding on the exposure zones in the flow passages. Thereafter, the flow rate is sequentially increased to flow rates of 0.5, 1, 2, 4, and 8 μl/sec. Each flow rate is maintained for 0.5-5 minutes, generally about one minute. An image is captured following each step to quantify the number of bound cells. Finally, a maximum flow rate is run for an additional 3-10 minutes, allowing firmly adhered cells to be quantified.

### Example 2

Using the results from Example 1, it is determined that a particular candidate compound enhances binding more than any other candidate compound. Thus, the procedure of Example 1 is repeated except eight samples containing eight different concentrations of the candidate compound are employed instead of eight samples each containing a different candidate compound.

We claim:

1. A device for exposing a substrate surface to one or more fluids, the device comprising:
a substrate having a surface containing a contiguous target region;
a cover plate having a surface capable of fluid-tight contact with the substrate surface;
a plurality of fluid-transporting features present in the cover plate surface, the fluid-transporting features separated by at least one partitioning wall representing an integral portion of the cover plate;
a plurality of inlets, wherein each inlet is in fluid communication with a fluid-transporting feature;
at least one outlet associated with the plurality of fluid-transporting features; and
a positioning means for positioning the cover plate surface in fluid-tight contact with the substrate surface such that the at least one partitioning wall contacts a location on the contiguous target region,
wherein each fluid-transporting feature, in combination with the substrate surface, forms a flow passage containing a distinct exposure zone on the target region such that each distinct exposure zone is downstream from the inlet in fluid communication therewith and upstream from the at least one outlet.

2. The device of claim 1, wherein the substrate is detachable from the cover plate.

3. The device of claim 1, wherein the substrate surface is substantially planar.

4. The device of claim 1, wherein the fluid-transporting features are channels.

5. The device of claim 4, wherein the features comprise eight channels.

6. The device of claim 4, wherein the channels are substantially parallel to each other.

7. The device of claim 6, wherein each channel defines a flow direction from an upstream terminus towards a downstream terminus and the flow directions of the channels are the same.

8. The device of claim 7, comprising a single outlet in fluid communication with all of the channels.

9. The device of claim 4, wherein the channels are the same size.

10. The device of claim 9, wherein the channels each have a width of about 0.1 to about 500 micrometers.

11. The device of claim 10, wherein the channels each have a width of about 200 to about 400 micrometers.

12. The device of claim 1, wherein each distinct exposure zone contains a cell.

13. The device of claim 12, wherein each distinct exposure zone contains a different type of cell.

14. The device of claim 12, wherein each distinct exposure zone contains a plurality of cells.

15. The device of claim 14, wherein each distinct exposure zone contains a plurality of different cells.

16. The device of claim 12, wherein the entire target region is covered with a plurality of cells.

17. The device of claim 16, wherein the plurality of cells forms a cellular monolayer.

18. The device of claim 12, wherein each distinct exposure zone further contains a cell-adhering substance.

19. The device of claim 12, wherein an array of cellular features is present on the target region.

20. The device of claim 19, wherein at least a portion of a cellular feature is interposed between the target region and the at least one partitioning wall.

21. The device of claim 1, wherein each inlet is in fluid communication with a source of fluid.

22. The device of claim 21, wherein each inlet is in fluid communication with a different source of fluid.

23. The device of claim 21, wherein each inlet is in fluid communication with the same source of fluid.

24. The device of claim 21, wherein at least one source of fluid contains a suspension of cells.

25. The device of claim 24, wherein the suspension contains cells of different types.

26. The device of claim 24, wherein the suspension contains cells of the same type.

27. The device of claim 21, wherein at least one source of fluid contains a body fluid.

28. The device of claim 21, wherein at least one source of fluid contains a candidate compound for interaction with a cell.

29. The device of claim 1, wherein the positioning means allows for the repositioning of the at least one partitioning wall to contact the contiguous target region at a different location.

30. The device of claim 29, wherein the positioning means allows for the rotational reorientation of the cover plate and the substrate surfaces.

31. A method for exposing a substrate surface to a plurality of cells, comprising:
(a) providing a substrate having a surface containing a contiguous target region; and
(b) maintaining a plurality of fluids each in contiguous laminar flow over the target region, wherein each fluid conveys a cell over a distinct exposure zone on the target region, thereby exposing the distinct exposure zone to the cell.

32. The method of claim 31, wherein the distinct exposure zones are defined at least in part by at least one partitioning wall contacting the contiguous target region.

33. The method of claim 31, wherein the distinct exposure zones are elongated.

34. The method of claim 31, wherein the distinct exposure zones are substantially parallel to each other.

35. The method of claim 34, wherein a fluid flows over each distinct exposure zone in the same direction.

36. The method of claim 34, wherein the distinct exposure zones are the same size.

37. The method of claim 31, wherein each fluid conveys a plurality of cells.

38. The method of claim 37, wherein the cells in each fluid are conveyed in a single file manner over a distinct exposure zone.

39. The method of claim 31, further comprising, after step (a) and before step (b), (a′) immobilizing a cell in each distinct exposure zone.

40. The method of claim 39, wherein each distinct exposure zone contains a different type of immobilized cell.

41. The method of claim 39, wherein the entire target region is covered with a plurality of cells.

42. The method of claim 41, wherein the plurality of cells forms a cellular monolayer.

43. The method of claim 39, wherein step (a′) comprises immobilizing the cells to form an array of cellular features on the target region.

44. The method of claim 43, wherein each of the cellular features is located in an exposure zone.

45. The method of claim 43, wherein each of the cellular features is located in a plurality of exposure zones.

46. The method of any of claims 39-45, further comprising during or after step (b), (c) detecting a cell-cell interaction in any of the distinct exposure zones, if present, as a result of the contact or proximity between a cell conveyed by a fluid and an immobilized cell.

47. A method for detecting cell-cell interactions comprising:
(a) providing a substrate having a surface containing a contiguous target region;
(b) immobilizing a plurality of cells in the contiguous target region;
(c) placing at least one partitioning wall in contact with the contiguous target region, thereby defining a plurality of distinct exposure zones on the target region;
(d) maintaining a plurality of fluids each in contiguous laminar flow over the target region, wherein each fluid conveys a cell over a distinct exposure zone, thereby exposing any immobilized cells in the distinct exposure zone to the cell conveyed by the fluid; and
(e) detecting a cell-cell interaction, if present, in any of the distinct exposure zones as a result of the contact or proximity between a cell conveyed by a fluid and an immobilized cell.

48. A method for exposing a substrate surface to a plurality of fluids, comprising:
(a) providing
a substrate having a surface containing a contiguous target region,
a cover plate having a surface capable of fluid-tight contact with the substrate surface,
a plurality of fluid-transporting features present in the cover plate surface, the fluid-transporting features separated by at least one partitioning wall representing an integral portion of the cover plate,
a plurality of inlets, wherein each inlet is in fluid communication with a fluid-transporting feature, and
at least one outlet associated with the plurality of fluid-transporting features,
(b) positioning the cover plate surface in fluid-tight contact with the substrate surface such that the at least one partitioning wall contacts the contiguous target region at a location and that each fluid-transporting feature, in combination with the substrate surface, forms a flow passage containing a distinct exposure zone on the target region such that each distinct exposure zone is downstream from the inlet in fluid communication therewith and upstream from the at least one outlet; and
(c) maintaining one or more fluids in laminar flow from one or more sources through the inlets over the target region such that the one or more fluids contact the exposure zones on the target region.

49. The method of claim 48, further comprising, after step (c), (d) maintaining at least one additional fluid in contiguous laminar flow over the target region, wherein the at least additional one fluid contacts at least one secondary exposure zones on the target region that is different from the distinct exposure zones formed in step (b), thereby exposing the at least one secondary exposure zones to the at least one additional fluid.

50. The method of claim 49, wherein step (d) comprises repositioning the cover plate surface in fluid-tight contact with the substrate surface such that the at least one partitioning wall contacts the contiguous target region at a different location and that the flow passages formed as a result of each fluid-transporting feature, in combination with the substrate surface contain a secondary distinct exposure zone on the target region.

51. The method of claim 50, wherein the cover plate surface is rotationally reoriented over the substrate surface.

52. The method of claim 51, wherein the cover plate surface is rotationally reoriented over the substrate surface by 60° to 120°.

53. The method of claim 52, wherein the cover plate surface is rotationally reoriented over the substrate surface by 90°.
